(19) United States
(12) Patent Application Publication
(10) Pub. No.: US 2023/0132835 A1
(43) Pub. Date: May 4, 2023

(54) ELECTRONIC THERMOMETER

(71) Applicant: AMOSENSE CO.,LTD, Cheonan-si, Chungcheongnam-do (KR)

(72) Inventor: Beom Jin KIM, Cheonan-si, Chungcheongnam-do (KR)

(73) Assignee: AMOSENSE CO.,LTD, Cheonan-si, Chungcheongnam-do (KR)

(21) Appl. No.: 17/917,794

(22) PCT Filed: Apr. 12, 2021
(86) PCT No.: PCT/KR2021/004590

§ 371 Date: Oct. 7, 2022

(30) Foreign Application Priority Data
Apr. 10, 2020 (KR) 10-2020-0043898

(51) Int. Cl. G01K 13/25; A61B 5/01; A61B 5/00

(52) U.S. Cl. CPC G01K 13/25; A61B 5/01; A61B 5/6833; A61B 2562/0271

(57) ABSTRACT

An electronic thermometer is provided. The electronic thermometer according to an exemplary embodiment of the present invention is for periodically measuring a body temperature by being attached to a body of a user, and comprises: at least one temperature sensor mounted on the one surface of a flexible circuit board; a memory unit for storing information obtained via the temperature sensor; a communication unit for externally transmitting the information stored in the memory unit; a control unit for controlling the operations of the temperature sensor, memory unit, and communication unit; a power supply unit arranged on the one surface of the flexible circuit board such that driving power can be provided; and a protection member surrounding the flexible circuit board such that external exposure of the temperature sensor, memory unit, communication unit, control unit, and power supply unit can be prevented.

### CROSS REFERENCE TO RELATED APPLICATIONS

This application is a National Phase Entry of International Application No. PCT/KR2021/004590, filed on Apr. 12, 2021, designating the United States, which is based upon and claims priority to Korean Patent Application 10-2020-0043898 filed on Apr. 10, 2020, the entire contents of which are incorporated herein by reference.

### TECHNICAL FIELD

The present invention relates to an electronic thermometer.

### BACKGROUND

Generally, a body temperature is measured using a thermometer. Such a thermometer accurately measures the body temperature only after a predetermined time elapses in a state in which the thermometer is attached to or in contact with a body, and the body temperature is measured once.

Accordingly, conventional thermometers have a limitation that it is impossible to continuously trace and monitor user's body temperatures.

The present invention is applied in consideration of the above limitation and directed to providing an electronic thermometer capable of periodically measuring a user's body temperature in a state in which the electronic thermometer is attached to a user's body, and then storing the measured results.

### SUMMARY OF THE INVENTION

One aspect of the present invention provides an electronic thermometer which is attached to a user's body to periodically measure a body temperature, the electronic thermometer including one or more temperature sensors mounted on one surface of a flexible circuit board, a memory part which stores information obtained through the temperature sensors, a communication part which transmits the information stored in the memory part to an outside, a control part which controls the temperature sensors, the memory part, and the communication part to be driven, a power supply part disposed on the one surface of the flexible circuit board to provide driving power, and a protection member which surrounds the flexible circuit board to prevent the temperature sensors, the memory part, the communication part, the control part, and the power supply part from being exposed to the outside.

The one or more temperature sensors may include a first temperature sensor for measuring a user's body temperature and a second temperature sensor for measuring an external temperature.

The communication part may include an antenna pattern pattern-formed on the flexible circuit board and a driver chip for driving the antenna pattern.

The electronic thermometer may further include a heat transfer member which may be electrically connected to the first temperature sensor through a via hole and mounted on a surface opposite to a surface of the flexible circuit board, on which the first temperature sensor is mounted, to be in direct contact with a user's skin.

The protection member may include an exposure hole formed to pass through the protection member in a region corresponding to the heat transfer member, and the heat transfer member may be exposed to the outside through the exposure hole.

The heat transfer member may be formed of a metal material.

The power supply part may be a printed battery disposed on the one surface of the flexible circuit board.

The protection member may be formed of a material having flexibility.

The electronic thermometer may further include one or more indicators.

The electronic thermometer may further include an adhesive member disposed on the one surface of the protection member.

The control part may measure a user's body temperature at regular time intervals through the temperature sensors and store measured information in the memory part.

According to the present invention, since an electronic thermometer can periodically measure and store a user's body temperature in a state in which the electronic thermometer is attached to a user's body, information of the user's body temperature can be traced and monitored.

### BRIEF DESCRIPTION OF THE DRAWINGS

FIG. 1 is a view illustrating an electronic thermometer according to one embodiment of the present invention.

FIG. 2 is a bottom view illustrating the electronic thermometer from which a release film is separated in FIG. 1.

FIG. 3 is a view illustrating an internal structure of the electronic thermometer shown in FIG. 1.

FIG. 4 is a cross-sectional view along line A-A of FIG. 3.

FIG. 5 is a view illustrating an internal structure of an electronic thermometer according to another embodiment of the present invention.

FIG. 6 is a block diagram illustrating the electronic thermometer according to one embodiment of the present invention.

FIG. 7 is a schematic view illustrating a body temperature tracing system using the electronic thermometer according to one embodiment of the present invention.

### DETAILED DESCRIPTION

Hereinafter, embodiments of the present invention will be described in detail with reference to the accompanying drawings in order for those skilled in the art to easily perform the present invention. The present invention may be implemented in several different forms and is not limited to the embodiments described herein. Parts irrelevant to descriptions are omitted in the drawings in order to clearly explain the present invention, and the same or similar parts are denoted by the same reference numerals throughout this specification.

An electronic thermometer 100 or 200 according to one embodiment of the present invention may be implemented in a patch type and attached to a user's body, may periodically measure a body temperature in a state of being attached to the user's body, and then store the measured result.

Through this, the electronic thermometer 100 or 200 according to one embodiment of the present invention may serve as a datalogger in which a user's body temperatures are stored after the user's body temperature is periodically measured. In this case, the user may be a target person of which information of a body temperature needs to be traced.

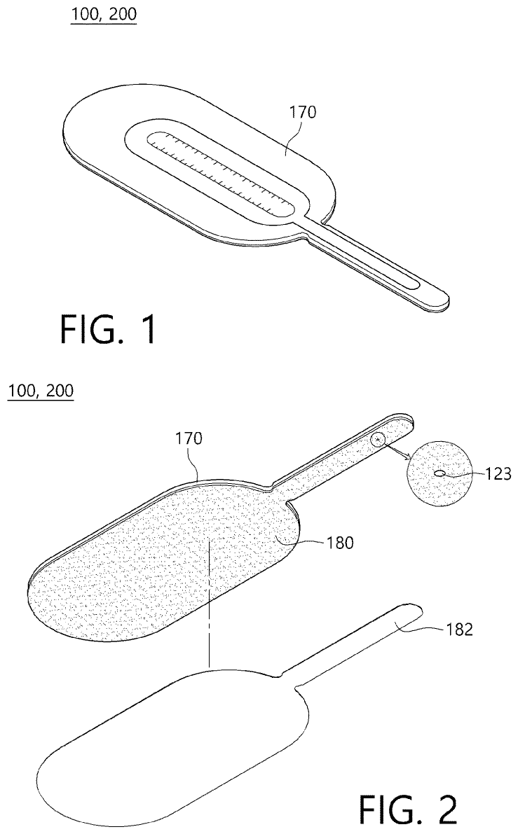

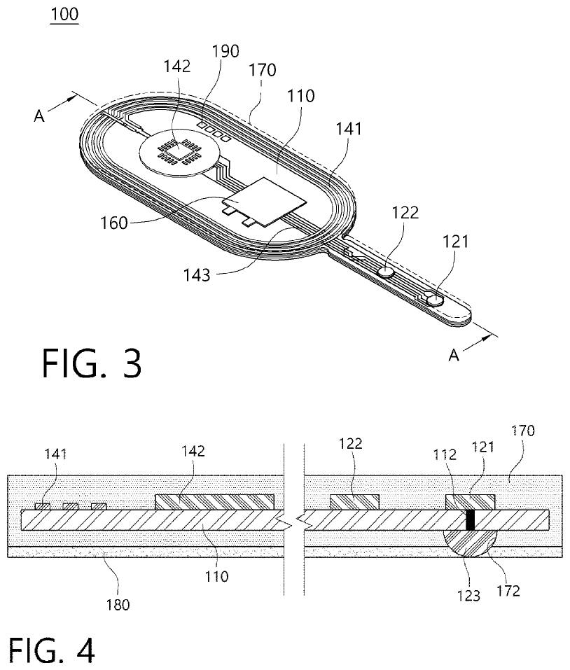

To this end, as illustrated in FIGS. 1 to 5, the electronic thermometer 100 or 200 according to one embodiment of the present invention includes a flexible circuit board 110, one or more temperature sensors 121 and 122, a memory part 130, a communication part 140, a control part 150, a power supply part 160, and a protection member 170.

The flexible circuit board 110 may be a board on which various circuit elements are mounted or a circuit pattern for electrical connection is formed. As an example, the circuit element may be a chipset for performing a predetermined function, and the circuit pattern may be an antenna pattern or a line pattern for electrical connection.

The flexible circuit board 110 may be a known flexible printed circuit board (FPCB) having flexibility and formed of polyimide (PI), polyethylene terephthalate (PET), or the like.

The one or more temperature sensors 121 and 122 may be mounted on one surface of the flexible circuit board 110 and may detect information about a temperature.

As an example, the one or more temperature sensors 121 and 122 may include a first temperature sensor 121 for detecting a user's body temperature to measure the user's body temperature. The first temperature sensor 121 may be mounted on one surface of the flexible circuit board 110 and may measure the user's body temperature at regular time intervals.

Here, information about the user's body temperature measured by the first temperature sensor 121 may be stored in the memory unit 130.

Through this, in a state in which the electronic thermometer 100 or 200 according to one embodiment of the present invention is attached to a user's skin, information about the body temperature periodically measured by the first temperature sensor 121 is periodically stored in the memory unit 130, all changes in the user's body temperature over time may be recorded, a checker may check result values for the body temperatures stored in the memory part 130, and thus a change in trend of the user's body temperature can be easily checked.

In this case, the first temperature sensor 121 may be a digital type temperature sensor. In addition, the first temperature sensor 121 may directly measure the user's body temperature, but may measure the user's body temperature based on heat transferred through a heat transfer member 123.

To this end, the first temperature sensor 121 may be mounted on a first surface of the flexible circuit board 110, and the heat transfer member 123 may be mounted on a second surface of the flexible circuit board 110 to be in direct contact with the user's skin. In this case, the first temperature sensor 121 and the heat transfer member 123 may be electrically connected to each other through a via hole 112 formed in the flexible circuit board 110.

Here, the first surface and the second surface of the flexible circuit board 110 may be opposite to each other. As an example, the first surface may be an upper surface of the flexible circuit board 110, and the second surface may be a lower surface of the flexible circuit board 110.

In addition, as described above, the heat transfer member 123 may be mounted on the second surface of the flexible circuit board 110 and may be in direct contact with the user's skin. Accordingly, a body temperature transferred from the user's skin may be transferred to the temperature sensors 121 and 122 through the heat transfer member 123.

As an example, the heat transfer member 123 may be formed of a metal material with high thermal conductivity.

In this case, the heat transfer member 123 may have a shape capable of always maintaining a state of being in contact with the user's skin when the electronic thermometer 100 or 200 according to one embodiment of the present invention is attached to the user's skin. To this end, the heat transfer member 123 may have a shape in which a central portion convexly protrudes in one direction.

As an example, the heat transfer member 123 may be formed in a hemispherical shape or dome shape.

Accordingly, when the electronic thermometer 100 or 200 according to one embodiment of the present invention is attached to the user's skin and even when an attached part is a curved body part, the central portion of the heat transfer member 123 may always maintain a state of being in contact with the user's skin. Accordingly, the heat transfer member 123 may easily transfer the heat transferred from the user's skin to the first temperature sensor 121.

As illustrated in FIGS. 2 and 4, the heat transfer member 123 may be exposed to the outside through an exposure hole 172 formed in the protection member 170 which will be described below. Accordingly, when the electronic thermometer 100 or 200 according to one embodiment of the present invention is attached to the user's body, the heat transfer member 123 may be always in direct contact with the user's skin.

However, an installation position of the first temperature sensor 121 is not limited thereto, and the first temperature sensor 121 may also be installed at a position at which the user's body temperature may be directly measured. As an example, the first temperature sensor 121 may be mounted on the second surface of the flexible circuit board 110 and exposed to the outside through the exposure hole 172 formed in the protection member 170. In this case, the heat transfer member 123 may be omitted.

In this case, the electronic thermometer 100 or 200 according to one embodiment of the present invention may measure an external temperature with the user's body temperature using the first temperature sensor 121.

To this end, the electronic thermometer 100 or 200 according to one embodiment of the present invention may further include a second temperature sensor 122 for measuring the external temperature in addition to the first temperature sensor 121 for measuring the user's body temperature.

The second temperature sensor 122 may be mounted on one surface of the flexible circuit board 110 like the first temperature sensor 121 and may measure the external temperature at regular time intervals. In this case, an exposure hole (not shown) may be formed in the protection member 170 in a region corresponding to the second temperature sensor 122, and the second temperature sensor 122 may also be exposed to the outside through the exposure hole.

Here, information about the external temperature measured by the second temperature sensor 122 may be stored in the memory part 130, and a detecting period of the second temperature sensor 122 may be a period which is the same as a detecting period of the first temperature sensor 121.

Through this, since the electronic thermometer 100 or 200 according to one embodiment of the present invention may measure temperature information about an external environment when the user's body temperature is measured in addition to information about the user's body temperature using the first temperature sensor 121, accuracy of the measured body temperature can be further improved.

As illustrated in FIG. 6, an overall operation of the one or more temperature sensors 121 and 122 may be controlled by the control part 150, the control part 150 may be driven using power provided by the power supply part 160, and information obtained by the one or more temperature sensors 121 and 122 may be stored in the memory part 130.

In this case, the one or more temperature sensors 121 and 122 may periodically detect a temperature at predetermined time intervals. As a non-restrictive example, the one or more temperature sensors 121 and 122 may measure a user's body temperature or an external temperature at every hour, and information about the measured temperature may be stored in the memory part 130.

Through this, since the electronic thermometer 100 or 200 according to one embodiment of the present invention minimizes consumption of power provided by the power supply part 160 while periodically measuring and storing the user's body temperature or the external temperature, a replacement period or recharge period of a battery can be increased.

The communication part 140 may transmit temperature information stored in the memory part 130 to an external electronic device through a wireless manner.

Here, the external electronic device 10 may be an electronic device in which a wireless communication module corresponding to the communication part 140 is installed. As an example, the external electronic device 10 may be a portable terminal 20, such as a smartphone, a repeater, a server, a cloud, or the like.

Accordingly, since the user or checker simply checks the temperature information stored in the memory part 130 using the external electronic device 10, the user or checker can easily check a change in trend of the user's body temperature. As an example, the communication part 140 may be a near field communication (NFC) antenna module. In this case, when the portable terminal 20 in which an NFC antenna module is installed is tagged on the communication part 140, the information stored in the memory part 130 may be transmitted to the portable terminal 20.

To this end, the communication part 140 may include an antenna pattern 141 pattern-formed on at least one surface of the flexible circuit board 110 and a driver chip 142 mounted on one surface of the flexible circuit board 110 to drive the antenna pattern 141.

Accordingly, the antenna pattern 141 may serve as a radiator configured to transmit information obtained through the one or more temperature sensors 121 and 122 to the outside through a wireless manner.

Through this, the temperature information measured by the one or more temperature sensors 121 and 122 may be transmitted to the external electronic device, for example, a portable terminal, through the antenna pattern 141. Accordingly, the user can simply check data measured by temperature sensors 121 and 122 through the portable terminal during the NFC tagging.

In this case, the data transmitted to the portable terminal through the antenna pattern 141 during the NFC tagging may be displayed by an application stored in the portable terminal. In addition, the data transmitted to the portable terminal through the antenna pattern 141 may also be stored in the application itself. Accordingly, the user can check the change in trend of the body temperature through the data stored in the application.

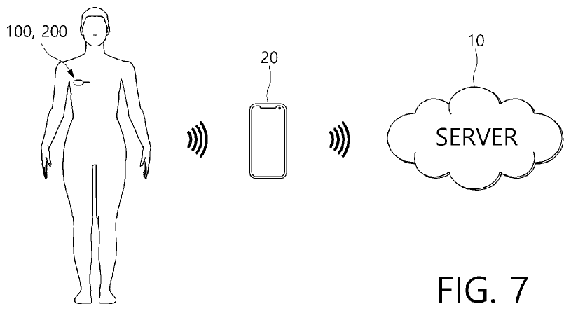

In addition, as illustrated in FIG. 7, the data transmitted to the portable terminal may be transmitted to the external electronic device 10, such as an additional server or cloud, using a network connected to the portable terminal 20 and then may be stored in the server or the external electronic device, and an instruction and an action according to a change in the body temperature may also be guided to the portable terminal 20 in conjunction with the server.

In this case, the driver chip 142 may be disposed inside the antenna pattern 141, the first temperature sensor 121 may be disposed outside the antenna pattern 141, and the driver chip 142 and the temperature sensors 121 and 122 may be electrically connected to each other through a lead part 143 formed on at least one surface of the flexible circuit board 110.

Accordingly, the first temperature sensor 121 does not need to be exposed to the user's skin and may be mounted on the first surface of the flexible circuit board 110 which is the same as a surface on which the driver chip 142 is mounted. Accordingly, since the first temperature sensor 121 may be completely covered by the protection member 170 which will be described below, airtightness can be improved.

As another example, the communication part 140 may be a Bluetooth module. In this case, the communication part 140 may be paired with the portable terminal 20 and may transmit the temperature information measured by the temperature sensors 121 and 122 to the portable terminal 20. That is, the temperature information measured by the temperature sensors 121 and 122 and stored in the memory part 130 may be transmitted to the external electronic device such as the portable terminal 20 through the Bluetooth module, and transmitted to and stored in an additional server or another electronic device 10 using a network connected to the external electronic device.

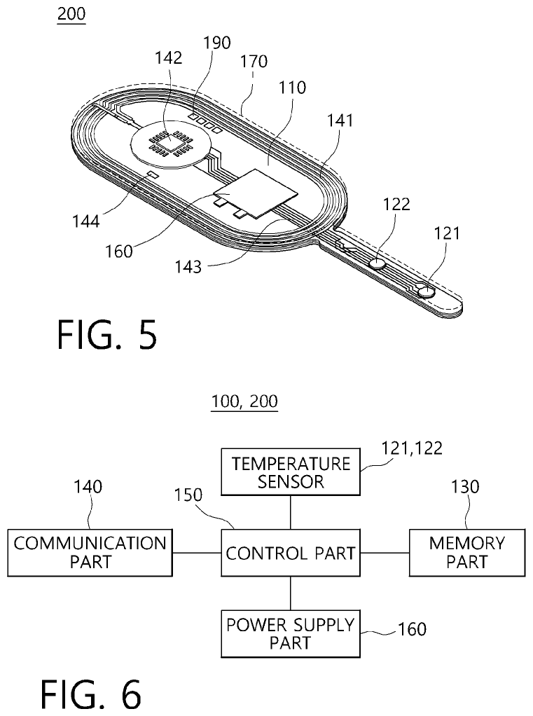

In addition, as illustrated in FIG. 5, the communication part 140 may also be formed in a form including all NFC antenna modules 141, 142, and 143, and the Bluetooth module 144.

The control part 150 may control an overall operation of the electronic thermometer 100 or 200 according to one embodiment of the present invention by controlling the temperature sensors 121 and 122, the memory part 130, and the communication part 140. The control part 150 may be driven using power provided by the power supply part 160.

In addition, the memory part 130 may serve as a storage medium configured to store the temperature information obtained through the one or more temperature sensors 121 and 122 as described above.

As a specific example, the control part 150 may be a system semiconductor such as a micro controller unit (MCU), and the memory part 130 may be a memory semiconductor such as a non-volatile memory (NVM).

In addition, although the power supply part 160 may be a known coin battery or prismatic battery, the power supply part 160 may be a flexible plate battery so that a capacity increases while an overall weight and volume are reduced. As a specific example, the power supply part 160 may be a known paper battery or printed battery.

That is, in the electronic thermometer 100 or 200 according to one embodiment of the present invention, since the power supply part 160 is implemented as the flexible plate battery, a power capacity for driving can be sufficiently secured, the overall weight can be reduced, and a thin film type can be implemented.

Meanwhile, the electronic thermometer 100 or 200 according to one embodiment of the present invention may include the protection member 170 which surrounds the flexible circuit board 110 to prevent the one or more temperature sensors 121 and 122, the memory part 130, the communication part 140, the control part 150, and the power supply part 160 from being exposed to the outside.

In this case, the protection member 170 may include the exposure hole 172 formed in a region corresponding to the heat transfer member 123 as described above, and the heat transfer member 123 may be exposed to the outside through the exposure hole 172.

Accordingly, the protection member 170 may be formed to completely surround portions excluding the heat transfer member 123.

That is, since the protection member 170 is formed to completely cover the upper surface and the lower surface of the flexible circuit board 110, the temperature sensors 121 and 122, the memory part 130, the communication part 140, the control part 150, the power supply part 160, and the flexible circuit board 110 excluding the heat transfer member 123 can be prevented from being exposed to the outside. In this case, the protection member 170 may be formed of a material having flexibility. Through this, even when the electronic thermometer 100 or 200 according to one embodiment of the present invention is attached to a curved body portion, since the electronic thermometer 100 or 200 may be smoothly deformed and fitted onto the user's curved body, adhesion with the user's body can be improved. As an example, the protection member 170 may be a molding part formed of an insulating resin material such as silicone. However, the protection member 170 is not limited thereto and may have a sheet type formed of a fluorine polymer resin such as PET, polypropylene (PP), or polyethylene (PE), release paper, or the like, and any material having insulation and airtightness can be used without limitation. Meanwhile, the electronic thermometer 100 or 200 according to one embodiment of the present invention may include an adhesive member 180 disposed on one surface of the protection member 170.

Since the adhesive member 180 has adhesiveness or viscosity, the adhesive member 180 can attach the electronic thermometer 100 or 200 to the user's body. Here, the adhesive member 180 may be disposed on a surface in which the exposure hole 172 for exposing the heat transfer member 123 to the outside is formed.

Accordingly, when the electronic thermometer 100 or 200 is attached to the user's skin through the adhesive member 180, the heat transfer member 123 may be in direct contact with the user's skin.

As an example, the adhesive member 180 may be an adhesive layer in a gel type and may be formed of a material of which an adhesive force is restored when coming into contact with moisture. Accordingly, the adhesive member 180 can be repeatedly reused.

As another example, the adhesive member 180 may be a known double-sided tape.

However, the material of the adhesive member 180 is not limited thereto, and any known material capable of providing an adhesive force with the user's skin may be used without limitation.

In addition, a removable release film 182 may be further attached to an exposure surface of the adhesive member 180.

Meanwhile, the electronic thermometer 100 or 200 according to one embodiment of the present invention may include at least one indicator 190.

The indicator 190 may display a remaining amount of the battery of the power supply part 160 or an on/off driving state.

As an example, the indicator 190 may be at least one light-emitting diode (LED) mounted on one surface of the flexible circuit board 110.

As described above, the electronic thermometer 100 or 200 according to one embodiment of the present invention may periodically measure a body temperature, store a measured result in the memory part 130, and transmit the result stored in the memory part 130 to the external electronic device through the communication part 140 in a state of being attached to the user's body. Through this, since the electronic thermometer 100 or 200 according to one embodiment of the present invention may be attached to a target person whose body temperature needs to be managed and may periodically measure body temperature information, a change in the body temperature of the body temperature management target person can be effectively traced.

In addition, when a body temperature greater than or equal to a predetermined temperature or a sudden change in body temperature is detected while the body temperature is periodically recorded through the first temperature sensor 121, the electronic thermometer 100 or 200 according to one embodiment of the present invention may transmit a warning signal to the external electronic device, such as the server, through the communication part 140, and a global positioning system (GPS) module may also be installed in the electronic thermometer 100 or 200 to check a position of the target person to which the electronic thermometer 100 or 200 is attached.

While embodiments of the present invention have been described above, the spirit of the present invention is not limited to the embodiments proposed in this specification, and other embodiments may be easily suggested by adding, changing and removing components by those skilled in the art and will fall within the spiritual range of the present invention.

1. An electronic thermometer which is attached to a user's body to periodically measure a body temperature, the electronic thermometer comprising:
one or more temperature sensors mounted on one surface of a flexible circuit board;
a memory part which stores information obtained through the temperature sensors;
a communication part which transmits the information stored in the memory part to an outside;
a control part which controls the temperature sensors, the memory part, and the communication part to be driven;
a power supply part disposed on the one surface of the flexible circuit board to provide driving power; and
a protection member which surrounds the flexible circuit board to prevent the temperature sensors, the memory part, the communication part, the control part, and the power supply part from being exposed to the outside.

2. The electronic thermometer of claim 1, wherein the one or more temperature sensors include:
a first temperature sensor for measuring a user's body temperature; and
a second temperature sensor for measuring an external temperature.

3. The electronic thermometer of claim 1, wherein the communication part includes:
an antenna pattern pattern-formed on the flexible circuit board; and
a driver chip for driving the antenna pattern.

4. The electronic thermometer of claim 2, further comprising a heat transfer member electrically connected to the first temperature sensor through a via hole and mounted on a surface opposite to a surface of the flexible circuit board, on which the first temperature sensor is mounted, to be in direct contact with a user's skin.

5. The electronic thermometer of claim 4, wherein:
the protection member includes an exposure hole formed to pass through the protection member in a region corresponding to the heat transfer member; and
the heat transfer member is exposed to the outside through the exposure hole.

6. The electronic thermometer of claim 4, wherein the heat transfer member is formed of a metal material.

7. The electronic thermometer of claim 1, wherein the power supply part is a printed battery disposed on the one surface of the flexible circuit board.

8. The electronic thermometer of claim 1, wherein the protection member is formed of a material having flexibility.

9. The electronic thermometer of claim 1, wherein the electronic thermometer is a digital temperature sensor further including at least one indicator.

10. The electronic thermometer of claim 1, further comprising an adhesive member disposed on the one surface of the protection member.

11. The electronic thermometer of claim 1, wherein the control part measures a user's body temperature at regular time intervals through the temperature sensors and stores measured information in the memory part.
